(19) United States
(12) Patent Application Publication
(10) Pub. No.: US 2024/0216455 A1
(43) Pub. Date: Jul. 4, 2024

(54) NON-STEROIDAL TOPICAL COMPOSITION AND METHOD THEREOF

(71) Applicant: ANUPAM GUPTA, Greater Noida (IN)

(72) Inventor: ANUPAM GUPTA, Greater Noida (IN)

(21) Appl. No.: 17/916,806

(22) PCT Filed: Jul. 23, 2021
(86) PCT No.: PCT/IN2021/050722

§ 371 Date: Dec. 15, 2022

(30) Foreign Application Priority Data
Jun. 9, 2021 (IN) 202111025676

(51) Int. Cl. A61K 36/31; A61K 9/00; A61K 33/04; A61K 33/14; A61K 33/42; A61K 36/54; A61K 36/9066

(52) U.S. Cl. CPC A61K 36/31; A61K 9/0014; A61K 33/04; A61K 33/14; A61K 33/42; A61K 36/54; A61K 36/9066

(57) ABSTRACT

The embodiments relate to a non-steroidal topical formulation comprising 10 wt % to 20 wt % of an active ingredient consisting of a combination of group I and group II component and 80 wt % to 90 wt % of inactive ingredient comprising one or more pharmaceutically acceptable excipient. The group I component maybe a plant extract obtained from Curcuma spp, Litsea spp., and Lepidium spp. and group II component may be selected from a group comprising Potassium sulfate (Kali sulphuricum), Potassium Phosphate (Kali phosphoricum), Sodium sulfate (Natrum sulphuricum, Sodium chloride (Natrum phosphoricum), Potassium Chloride (Kali muriaticum), and Sodium Phosphate (Natrum phosphoricum). The said formulation is capable of efficiently eliminating or reducing age-related osteoarthritic joint pain, inflammation and musculoskeletal diseases and provides long-term relief to the aged patients. Another preferred embodiment of the present invention relates to a method of preparing the said formulation.

### FIELD OF THE INVENTION

The present invention generally relates to a non-steroidal topical formulation and method thereof. Specifically, the present invention relates to a novel and improved non-steroidal topical formulation and method of preparing the same for eliminating or reducing age-related osteoarthritic joint pain, inflammation and musculoskeletal diseases in a human subject.

### BACKGROUND OF THE INVENTION

Majority of the old-aged people develop knee-joint pain due to the wear and tear in the joints. Arthritis is the inflammation of one or more joints and osteoarthritis is the most common form of arthritis, affecting millions of people worldwide. It is a degenerative joint disease that is caused due to the deterioration of the cartilage in joints, resulting in inflammation, pain and soreness in the patients. It occurs with the normal process of aging or it can be heredity. Any injury from trauma or disease may also lead to osteoarthritis.

Treatments available to relieve such pain include administration of local painkillers (ointments or oil) which work for a short duration. Considerable efforts have been made to provide long term relief from chronic joint pain and inflammation which includes medical devices, oral painkiller formulation, topical ointments, pain relief patches or similar kind of treatments but such treatments give temporary relief to the patients and ultimately they will have to shift to oral painkillers which induce multiple side effects ranging from acidity to dyspepsia, etc. Long-term treatment with oral Nonsteroidal anti-inflammatory drugs or NSAIDs can result in stomach problems like bleeding, ulcer, and stomach upset. Lastly, patients have to go for a total joint replacement surgery which has its own limitations and drawbacks. Such formulations and treatments to reduce the pain and inflammation associated with osteoarthritis are known in the art, for instance

U.S. Pat. No. 5,827,886A relates to a topical composition to reduce chronic pain and inflammation. Such composition includes reduced glutathione, a selenoamino acid and an anesthetic, such as capsaicin, in a suitable carrier for topical application. However, the composition as disclosed in the cited art fails to provide long-term pain relief to the patients.

U.S. Pat. No. 8,105,624B2 relates to a topical patch comprising a drug N,2,3-Trimethyl-2-isopropylbutamide, adhesive gel composition and insoluble support. However, such a topical patch fails to provide long-term relief to patients with chronic joint pain and inflammation.

Therefore, considering the above shortcomings with the existing formulations and treatments which provides temporary and short term relief to the patients, there is a need for a topical formulation that can provide long term relief similar to the oral medications from the joint pain, inflammation and musculoskeletal diseases in aged patients who are suffering from any type of arthritis, particularly osteoarthritis in aged people.

### Object of the Invention

An object of the present invention is to provide a novel and improved topical formulation for eliminating or reducing osteoarthritic joint pain, Inflammation and musculoskeletal diseases in a human subject and method of preparing the same. In particular, the present invention relates to a non-steroidal topical formulation that may provide long-term relief in age-related osteoarthritic joint pain, inflammation and musculoskeletal diseases in a human subject.

Another object of the present invention is to provide a non-steroidal topical formulation that may provide long-term relief in a human subject and it may help in reducing the intake of oral medications.

Another object of the present invention pertains to providing a method of preparing the said topical formulation.

### SUMMARY OF THE INVENTION

The present invention is described hereinafter by various embodiments. This invention may, however, be embodied in many different forms, should not be construed as limited to the embodiments set forth herein. Rather, the embodiments are provided with the intent of imparting clarity pertaining to the scope of the invention to those skilled in the art.

In an embodiment, the present invention overcomes the existing problems by providing a novel and improved non-steroidal topical formulation which may provide long-term relief in age-related osteoarthritic pain, inflammation and musculoskeletal diseases in a human subject. The invention also provides a method of preparing the said topical formulation.

In a preferred embodiment, the present invention relates to a non-steroidal topical formulation and method thereof for eliminating or reducing age-related osteoarthritic joint pain, inflammation and musculoskeletal diseases in a human subject. The non-steroidal topical formulation comprises an active ingredient consisting of a combination of group I and group II component and an inactive ingredient comprising at least one pharmaceutically acceptable excipient. The group I component maybe a plant extract obtained from Curcuma spp, Litsea spp., and Lepidium spp. and group II component may be selected from a group comprising Potassium sulfate (Kali sulphuricum), Potassium Phosphate (Kali phosphoricum), Sodium sulfate (Natrum sulphuricum, Sodium chloride (Natrum muriaticum). Potassium Chloride (Kali muriaticum), and Sodium Phosphate (Natrum phosphoricum).

In another preferred embodiment, the present invention discloses a method of preparing the said formulation.

### DETAILED DESCRIPTION OF THE INVENTION

Various modifications to these embodiments are apparent to those skilled in the art from the description and the accompanying drawings. The principles associated with the various embodiments described herein may be applied to other embodiments. Therefore, the description is not intended to be limited to the embodiments shown with the accompanying drawings but is to provide broadest scope consistent with the principles and the novel as well as inventive features disclosed or suggested herein. Accordingly, the invention is anticipated to hold on to all other alternatives, modifications, and variations that fall within the scope of the present invention.

The invention discloses a novel and improved non-steroidal topical formulation and method thereof. The present invention aims to provide a formulation that may be used in eliminating or reducing age-related osteoarthritic joint pain, inflammation and musculoskeletal diseases and provides long-term relief without the use of any oral medication. Here the “age-related osteoarthritic pain” refers to the occurrence of osteoarthritis in older people including men and women who are above the age of 50 Here “long-term relief” refers to the efficacy of the formulation in reducing the pain at the application site as upon application initially, the formulation works for a variable duration ranging from 6 to 8 hours and later it extends from 12 to 24 hours. Therefore, the patient gets relief from pain for a longer duration

### Non-Steroidal Topical Formulation

In a preferred embodiment, the non-steroidal topical formulation according to the invention comprises 10 wt % to 20 wt % of an active ingredient consisting of a combination of group I and group II component and 80 wt % to 90 wt % of inactive ingredient comprising one or more pharmaceutically acceptable excipient. The group I component maybe a plant extract obtained from Curcuma spp. Litsea spp., and Lepidium spp. and group II component may be selected from a group comprising Potassium sulfate (Kali sulphuricum), Potassium Phosphate (Kali phosphoricum). Sodium sulfate (Natrum sulphuricum), Sodium chloride (Natrum muriaticum), Potassium Chloride (Kali muriaticum), and Sodium Phosphate (Natrum phosphoricum). The formulation particularly helps in eliminating or reducing age-related osteoarthritic joint pain, inflammation and musculoskeletal diseases and provides long-term relief in patients above the age of 50.

In one embodiment, the present invention emphasize on a combination of active and inactive ingredients in the said topical formulation. It is pertinent to note that the active ingredients that are added to the formulation act as a pain killer which efficiently eliminates or reduces age-related osteoarthritic joint pain, inflammation and musculoskeletal diseases and provides long-term relief to aged patients. The active ingredient is present in the range of 10 wt % to 20 wt % and it is divided into group I and group II components. The two components of the formulation must be present in a specific amount so that the active ingredient gets absorbed into the surface of the skin of the patient where it is locally applied. Additionally, the active ingredient must penetrate the outer protective barrier of the skin and reach the viable lower layers of the epidermis and dermis to effectively treat pain for a long duration.

### Active Ingredients

In an exemplary embodiment, the present invention may evaluate the analgesic and anti-inflammatory effect of the plant extracts present in a specific amount along with other components in eliminating or reducing age-related joint pain, inflammation and musculoskeletal diseases and provides long-term relief in patients suffering from osteoarthritis.

The plant extracts used in the topical formulation may be selected from powdered plant parts of Curcuma spp, Litsea spp, and Lepidium spp. Preferably, C. aromatica, Lepidium sativum and Tallow Soft Bollygum Laurel Litsea Glutinosa. Particularly, the group I component of the topical formulation comprises 30 wt % to 35 wt % of plant extract obtained from Curcuma spp. 30 wt % to 35 wt % of plant extract obtained from Litsea spp and 30 wt % to 35 wt % of plant extract obtained from Lepidium spp. The plant extracts may be obtained from plant parts selected from rhizome, seed and wood. Preferably, group I component comprises 30 wt % to 35 wt % of plant extract obtained from rhizome of Curcuma spp. 30 wt % to 35 wt % of plant extract obtained from woods of Litsea spp and 30 wt % to 35 wt % of plant extract obtained from seeds of Lepidium spp.

Curcuminoids are natural polyphenol compounds derived from Curcuma spp. or turmeric, which is a member of the ginger family (Zingiberaceae). These compounds are known for their anti-inflammatory, antithrombotic, antioxidant, and antimicrobial activities. It may be observed that Curcumin-containing products demonstrate significant improvement in treating osteoarthritis. Accordingly, in the present invention plant extracts from Curcuma spp., which may include, but are not limited to, C. aromatica, C. amada, C. longa, or C. domestica. The plant from Curcuma spp is commonly known as Haldi.

Lepidium sativum Linn., commonly known as “garden cress,” or “halo meda” belongs to Cruciferae family. Litsea glutinosa commonly known as “meda lakari” is a rainforest tree in the laurel family, Lauraceae. It is further contemplated that the preferred embodiment may also include other active ingredients selected from a group comprising Alpinia galanga commonly named as rasna patra, Castor seed or commonly known as arandi beej. Wintergreen oil commonly known as gandhpura oil, turpentine oil commonly known as tarpin oil, Eucalyptus oil commonly known as Nilgiri oil. Vitex negundo commonly known as Nirgundi oil. Chilli or Red Pepper oil commonly known as Katuvira oil and methanol.

In another embodiment, the group II components may comprise homeopathic compounds. The group II components may include one or more homeopathic active ingredients selected from a group comprising Potassium sulfate (Kali sulphuricum), Potassium Phosphate (Kali phosphoricum). Sodium sulfate (Natrum sulphuricum), Sodium chloride (Natrum muriaticum). Potassium Chlonde (Kali muriaticum), and Sodium Phosphate (Natrum phosphoricum). Preferably, the amount of Potassium Phosphate (Kali phosphoricum) is in the range of 85 wt % to 95 wt %, Potassium sulfate (Kali sulphuricum) is in the range of 5 wt % to 15 wt %, Sodium sulfate (Natrum sulphuricum) is in the range of 5 wt % to 15 wt %, Sodium chloride (Natrum muriaticum) is in the range of 5 wt % to 15 wt %. Potassium Chloride (Kali muriaticum) is in the range of 5 wt % to 15 wt %, and Sodium Phosphate (Natrum phosphoricum) is in the range of 5 wt % to 15 wt %. It is also important to maintain the potency of the group II components. Here the word “potency” refers to the degree of dilution of homeopathic medicine. In homeopathy, potency is inversely related to concentration, the greater the dilution the higher the potency of the homeopathic remedy. According to the HPUS, it may be quantified according to various scales, such as the decimal X scale, centesimal C scale and quintamillesimal Q scale. In general, a decimal X scale dilution is half the value of a C scale dilution, and a given dilution on the Q scale is about 2.35 times the value of a C scale dilution. In the present invention, the potency of the group II components is 200/200x.

In yet another embodiment, it is contemplated that the formulation of the present invention may be further mixed with other conventional active ingredients used in an ointment that is known to act as a painkiller. Thus, the unique formulation can impart long-acting effects which relieve osteoarthritis patients of severe knee pain thereby replacing the need of using oral pain killers.

### Inactive Ingredients

In another embodiment, the present invention relates to the inactive ingredients. Here the “inactive ingredients” refer to one or more pharmaceutically acceptable excipients which may include, but are not limited to bases, vehicles, and solvents. The inactive ingredients may influence the quality attributes of topical formulation such as physicochemical characteristics of the drug, and sensorial characteristics (i.e., smell, texture, cooling and burning effect, absorption, etc) of the formulation. However, it is pertinent to note that the inactive ingredients may ultimately affect the final performance of the topical formulation by affecting properties such as solubility, stability, release of the active ingredient, and skin penetration.

In yet another embodiment, the inactive ingredients are present in a therapeutically effective amount in the topical formulation comprising 80 wt % to 90 wt % of the formulation. Here the term “therapeutically effective amount” of the inactive ingredient as used herein, is an amount that is effective for the prevention and/or treatment of an ailment or injury in a mammal (preferably a human), without undue adverse side effects (such as toxicity, irritation, or allergic response), commensurate with a reasonable benefit/risk ratio when used in the manner of this invention. The inactive ingredient may act as a topical analgesic agent, polyacrylic acid polymer, emollient, chelating agent, pH adjuster, antioxidant, emulsifying wax and cooling agent.

In an exemplary embodiment, the present invention discloses methyl salicylate as a topical analgesic agent, Carbopol-940 as a polyacrylic acid polymer, Glycerin as an emollient, Di Sodium ethylenediamine tetraacetic acid (Di-EDTA) as a chelating agent, Triethanolamine (TEA) as a pH adjuster, Butylated hydroxytoluene (BHT) as an antioxidant. Glyceryl monostearate as an emulsifying wax, Cetyl Alcohol as a cooling agent or counter-irritant and water as a solvent.

### Method of Preparing the Formulation

In a preferred embodiment, the present invention relates to a method of preparing a non-steroidal topical formulation wherein the steps may comprise
i. taking plant extract obtained from Curcuma spp in the range of 30 wt % to 35 wt %, from Litsea spp. in the range of 30 wt % to 35 wt %, and from Lepidium spp. in the range of 30 wt % to 35 wt % to obtain group I component;
ii. taking 5 wt % to 15 wt % of Potassium sulfate (Kali sulphuricum), 85 wt % to 95 wt % of Potassium Phosphate (Kali phosphoricum), 5 wt % to 15 wt % of Sodium sulfate (Natrum Sulphuricum), 5 wt % to 15 wt % of Sodium chloride (Natrum muriaticum), 5 wt % to 15 wt % of Potassium Chloride (Kali muriaticum), and 5 wt % to 15 wt % of Sodium Phosphate (Natrum phosphoricum) to obtain group II component;
iii. mixing the group I and group II components to obtain an active ingredient;
iv. adding 10 wt % to 20 wt % of the active ingredient obtained from step (iii) with 80 wt % to 90 wt % of inactive ingredient along with to obtain the topical formulation; and
   wherein the group I and group II component are present in the ratio of 1:1.

### Application and Effectivity

The formulation of the present invention may be intermittently or continuously reapplied as necessary to provide either a continuous dosage or multiple dosages over time. It may be effective for as long as 6 to 8 hours a day from the time of application. It may be applied gently on the skin over the affected area to facilitate penetration. In case of knee pain, it is applied around the knee including the outer, inner, front and back sides in intervals of 6 hours. With time, the effectiveness of the formulation increases to at least 24 hours even when applied once or twice a day.

It will be recognized by those skilled in the art that the formulations and treatments described herein are effective in treating humans suffering from age-related osteoarthritic joint pain or knee pain, inflammation and musculoskeletal diseases and provides long-term relief to the aged patients.

It should be understood that the topical formulation of the present invention may comprise any dosage form suitable for delivery of the formulation to an affected site where pain and/or inflammation is established or is anticipated. Non-limiting examples of dosage forms that may incorporate the said non-steroidal topical formulation may include gels, including hydrogels, creams, ointments, salves, balms, lotions, liniments, cream gels, lotion ointments, spray and decoctions and combinations thereof.

It will be readily understood that the components of various embodiments of the present invention, as generally described and illustrated in the FIGURES herein, may be arranged and designed in a wide variety of different configurations. Thus, the detailed description of the embodiments of the present invention, as represented in the attached FIGURES, is not intended to limit the scope of the invention as claimed, but is merely representative of selected embodiments of the invention.

The features, structures, or characteristics of the invention described throughout this specification may be combined in any suitable manner in one or more embodiments. For example, reference throughout this specification to “certain embodiments,” “some embodiments,” or similar language means that a particular feature, structure, or characteristic described in connection with the embodiment is included in at least one embodiment of the present invention. Thus, appearances of the phrases “in certain embodiments,” “in some embodiment,” “in other embodiments,” or similar language throughout this specification do not necessarily all refer to the same group of embodiments and the described features, structures, or characteristics may be combined in any suitable manner in one or more embodiments.

It should be noted that reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

One having ordinary skill in the art will readily understand that the invention as discussed above may be practiced with steps in a different order, and/or with hardware elements in configurations that are different than those which are disclosed. Therefore, although the invention has been described based upon these preferred embodiments, it would be apparent to those of skill in the art that certain modifications, variations, and alternative constructions would be apparent while remaining within the spirit and scope of the invention. In order to determine the metes and bounds of the invention, therefore, reference should be made to the appended claims.

### Advantages of the Present Invention

The formulation is effective in eliminating or reducing age-related osteoarthritic joint pain, inflammation and musculoskeletal diseases in older patients
The formulation provides extensive relief in osteoarthritis of the knee joint.
It provides long-term relief i.e. for 6 to 8 hours which later extends from 12 to 24 hours, without the administration of any oral medication and side effects.
The formulation may be added to any conventional formula to provide novel and invention
The formulation is novel, efficient and inexpensive.

### Examples

The group I components were obtained from Haridwar. India and the group II components were commercially purchased from Noida. India
A. Active ingredients: The active ingredients were divided into two groups I and II.

### Group I Components

TABLE 1

| | | | Amount | |
|---|---|---|---|---|
| Name | Family | Part used | (in wt %) | Function |
| Curcuma spp | Zingiberaceae | Rhizome | 30-35 wt % | Painkiller |
| Litsea spp. | Lauraceae | Wood | 30-35 wt % | Painkiller |
| Lepidium spp. | Cruciferae | Seeds | 30-35 wt % | Painkiller |

The above table 1, demonstrates the details of the active ingredients falling in group I of the topical formulation.

### Group II Components

TABLE 2

| English | Commercial name | Amount | | |
|---|---|---|---|---|
| Name | (as per *HPUS) | (in wt %) | Potency | Inactive |
| Potassium | Kali | 1-5 | 200/200x | In base of |
| sulfate | sulphuricum | | | Acacia Gum |
| | | | | and Lactose |
| Potassium | Kali | 85-95 | 200/200x | In base of |
| phosphate | phosphoricum | | | Acacia Gum |
| | | | | and Lactose |
| Sodium | Natrum | 1-5 | 200/200x | In base of |
| sulfate | sulphuricum | | | Acacia Gum |
| | | | | and Lactose |
| Sodium | Natrum | 1-5 | 200/200x | In base of |
| chloride | muriaticum | | | Acacia Gum |
| | | | | and Lactose |
| Potassium | Kali | 1-5 | 200/200x | In base of |
| Chloride | muriaticum | | | Acacia Gum |
| | | | | and Lactose |
| Sodium | Natrum | 1-5 | 200/200x | In base of |
| Phosphate | phosphoricum | | | Acacia Gum |
| | | | | and Lactose |

*HPUS: Active ingredients are in the official homeopathic pharmacopeia of USA

The above table 2, demonstrates the details of the active ingredients falling in group II of the topical formulation.
B. Inactive ingredients: The inactive ingredients act as stabilizers or provide other functions like fragrance etc. These inactive ingredients either help active ingredients at different stages to avoid its disintegration or provide features that will be accepted by patients (good fragrance, cooling and burning effect, absorption and avoid coloring on use and similar benefits).

What is claimed is:

1. A non-steroidal topical formulation comprising:
10 wt % to 20 wt % of an active ingredient consisting of a combination of group I and group II component; and
80 wt % to 90 wt % of inactive ingredient comprising one or more pharmaceutically acceptable excipient:
wherein group I component is a plant extract obtained from Curcuma spp. Litsea spp., and Lepidium spp. and group II component is selected from a group comprising Potassium sulfate (Kali sulphuricum), Potassium Phosphate (Kali phosphoricum), Sodium sulfate (Natrum sulphuricum), Sodium chloride (Natrum muriaticum), Potassium Chloride (Kali muriaticum), and Sodium Phosphate (Natrum phosphoricum).

2. The formulation as claimed in claim 1, wherein the plant extract is obtained from rhizome, wood and seeds of a plant.

3. The formulation as claimed in claim 1, wherein the group I component comprises 30 wt % to 35 wt % of plant extract obtained from Curcuma spp, 30 wt % to 35 wt % of plant extract obtained from Litsea spp and 30 wt % to 35 wt % of plant extract obtained from Lepidium spp.

4. The formulation as claimed in claim 1, wherein the group II component comprises 85 wt % to 95 wt % of Potassium Phosphate (Kali phosphoricum), 5 wt % to 15 wt % of Potassium sulfate (Kali sulphuricum), 5 wt % to 15 wt % of Sodium sulfate (Natrum sulphuricum), 5 wt % to 15 wt % of Sodium chloride (Natrum muriaticum), 5 wt % to 15% of Potassium Chloride (Kali muriaticum), and 5 wt % to 15 wt % of Sodium Phosphate (Natrum phosphoricum).

5. The formulation as claimed in claim 1, wherein the inactive ingredient is selected from a group comprising topical analgesic agent, polyacrylic acid polymer, emollient, chelating agent, pH adjuster, antioxidant, emulsifying wax, cooling agent and solvent.

6. The formulation as claimed in claim 1, wherein the formulation is in the form of an ointment, cream, lotion, gel and spray.

7. The formulation as claimed in claim 1, wherein the formulation is effective in reducing age-related osteoarthritic joint pain and musculoskeletal diseases.

8. A method of preparing a topical formulation, wherein the method comprises
(i) taking plant extract obtained from Curcuma spp in the range of 30 wt % to 35 wt %, from Litsea spp. in the range of 30 wt % to 35 wt %, and from Lepidium spp. in the range of 30 wt % to 35 wt % to obtain group I component;
(ii) taking 5 wt % to 15 wt % of Potassium sulfate (Kali sulphuricum), 85 wt % to 95 wt % of Potassium Phosphate (Kali phosphoricum), 5 wt % to 15 wt % of Sodium sulfate (Natrum sulphuricum), 5 wt % to 15 wt % of Sodium chloride (Natrum muriaticum), 5 wt % to 15 wt % of Potassium Chloride (Kali muriaticum), and 5 wt % to 15 wt % of Sodium Phosphate (Natrum phosphoricum) to obtain group II component;
(iii) mixing the group I and group II components to form an active ingredient;
(iv) adding 10 wt % to 20 wt % of the active ingredient obtained from step (iii) with 80 wt % to 90 wt % of inactive ingredient along with to obtain the topical formulation; and
wherein the group I and group II component are mixed in the ratio of 1:1.
